(19) United States
(12) Patent Application Publication
(10) Pub. No.: US 2013/0116408 A1
(43) Pub. Date: May 9, 2013

(54) Virion Derived Protein Nanoparticles For Delivering Radioisotopes For The Diagnosis And Treatment Of Malignant And Systemic Disease And The Monitoring Of Therapy

(71) Applicant: Elisabet de los Pinos, Brookline, MA (US)

(72) Inventor: Elisabet de los Pinos, Brookline, MA (US)

(73) Assignee: AURA Biosciences, Inc., Cambridge, MA (US)

(21) Appl. No.: 13/469,934

(22) Filed: May 11, 2012

Related U.S. Application Data
(63) Continuation-in-part of application No. 13/367,296, filed on Feb. 6, 2012.
(60) Provisional application No. 61/556,218, filed on Nov. 5, 2011.
(60) Provisional application No. 61/567,074, filed on Dec. 5, 2011.

(51) Int. Cl. C12P 21/00; C07K 14/00

(52) U.S. Cl. 530/350; 435/69.1

(57) ABSTRACT

The invention is directed to novel compositions and methods utilizing virion derived protein nanoparticles for delivery of medical imaging agents and therapeutic agents for the diagnosis and treatment of malignant and systemic diseases. The nanoparticles of the present invention are designed to deliver radioactive isotopes suitable for imaging a tumor and its metastases. Additionally, the nanoparticles may deliver a radioisotope that is suitable for treating a tumor and its metastases by alpha, beta or gamma radiation. Alternatively, the virion derived nanoparticles may deliver a treatment agent for cancer or a combination of a radioisotope and a cancer treatment agent. Additionally the virion derived nanoparticle may include delivery of a drug that enhances the immune system's recognition of the tumor.

### RELATED APPLICATIONS

The present application is a Continuation In Part (CIP) of U.S. patent application Ser. No. 13/367,296 filed Feb. 6, 2012 which claims the benefit of priority to U.S. Provisional Application No. 61/556,218 filed Nov. 5, 2011 and U.S. Provisional Application No. 61/567,074 filed Dec. 5, 2011. The disclosures of the above applications are incorporated herein by reference.

### FIELD OF INVENTION

The invention relates to novel compositions and methods for diagnosing and treating malignant diseases by delivering radioisotope loaded protein nanoparticles to tumor cells.

### REFERENCE TO SEQUENCE LISTINGS

The Sequence Listing provides exemplary polynucleotide sequences of the invention. The traits associated with the used of the sequences are included in the Examples.

The Sequence Listing submitted as an initial paper is named AURA_{—}18C_Sequence Listing_ST25.txt, is 16.0 kilobytes in size, and the Sequence Listing was created on 29Jan. 2012. The copies of the Sequence Listing submitted via EFS-Web as the computer readable for are hereby incorporated, by reference in their entirety.

### BACKGROUND OF THE INVENTION

For over sixty years, radioisotopes have been used in medicine for diagnostic and imaging applications. To detect a tumor in an organ, radioactive drugs are administered to a patient through inhalation, injection, or orally. These uses of radioisotopes have led to numerous applications for imaging specific organs and larger functions within the body. However, the utility of regional radionuclide treatments is limited by the fact that such strategies are effective only in the chosen treatment volume; they cannot be used to treat tumor metastases. This obstacle could be overcome by developing true tumor-seeking agents that selectively deliver radionuclides to cancer cells regardless of where they are located

One of the main limitations of ultra-potent radioisotopes like alpha emitters and auger emitters is the short distance they travel (of the order of 1 nm to 1 μm), thus their biological effects are highly dependent upon their cellular and sub-cellular distribution. For cancer treatment using the cell nucleus and the plasma membrane as examples, the published results indicate that the electron dose rate delivered to the nucleus is between 18 to 94 times higher if the radionuclides are localized within the cell nucleus than if they are situated only on the cell membrane. (Hofer et al, Acta Oncologica Vol. 35, No. 1, pp. 189-196, 1996).

To improve treatment options using radioisotopes, researchers have started experimenting with the use of virus like nanoparticles (VLPs) to encapsulate deliver radioisotopes to specific cells within the body. For instance, Roberts et al., US Pub. No. US2010/0135902A1, discusses the use of papillomavirus VLPs for the detection and therapy of tumors.

Further, several additional researchers have described various systems to create VLPs for use as delivery vehicles for a variety of treatments. For example, HPV L1 and/or L2 VLPs have been generated in Spodoptera frugiperla (Sf21) cells. Baculoviruses encoding the L1 and/or L2 gene of HPV of different serotypes (e.g., HPV16, HPV18, HPV31, and HPV58) have been described in Touze et al., FEMS Microbiol. Lett. 2000; 189:121-7; Touze et al., J. Clin. Microbiol. 1998; 36:2046-51); and Combita et al., FEMS Microbiol. Lett. 2001; 204(1): 183-8. Additionally, viral capsid proteins have also been created using galactose-inducible Saccharomyces cerevisiae expression system. An exemplary protocol can be found in Cook et al. Protein Expression and Purification 17, 477-484 (1999).

Further, Buck et al. (J. Virol. 78, 751-757, 2004) reported the production of papilloma virus-like particles (VLP) and cell differentiation-independent encapsidation of genes into bovine papillomavirus (BPV) L1 and L2 capsid proteins expressed in 293TT human embryonic kidney cells which stably expresses SV40 large T antigen to enhance replication of SV40 origin-containing plasmids. Also, Pyeon et al. reported a transient transfection method that achieved the successful and efficient packaging of full-length HPV genomes into HPV16 capsids to generate virus particles (PNAS 102, 9311-9316 (2005)).

However, despite the variety of methods for creating and loading VLPs which are currently under investigation, there does not presently exist a usable, safe and effective method for producing and administering VLPs loaded with radioisotopes for the treatment of cancer or other diseases. The primary obstacles to creating such treatments are based on the limitations of the virus particles themselves. More specifically, conventional VLPs are ineffective due to their inability to evade the body's immune system. Further, they are ineffective due their inability to deliver radioisotopes near the nucleus of a cell which is where they must be to damage the functions of the cell. The present systems for creating and loading VLPs fail to create particles which overcome these limitations.

### SUMMARY OF INVENTION

The object of the present invention is to overcome the shortcomings disclosed in the prior art. The present invention provides compositions and methods for the use of virion derived nanoparticles for delivering medical imaging agents and therapeutics in the field of nuclear medicine. In particular, the present invention provides protein based virus-like nanoparticles for the delivery of radioisotopes to primary tumor cells and metastases.

The nanoparticles of the present invention are designed to deliver radioactive isotopes suitable for imaging a tumor and its metastases. Additionally, the nanoparticles may deliver a radioisotope that is suitable for treating a tumor and its metastases by alpha, beta or gamma radiation. Alternatively, the virion derived, nanoparticles may deliver a treatment agent for cancer or a combination of a radioisotope and a cancer treatment agent. Additionally the virion derived nanoparticle may include delivery of a drug that enhances the immune system's recognition of the tumor.

More specifically, the present invention describes the use of recombinant proteins that mimic specific viruses. The novel virion derived protein nanoparticles can be efficiently loaded with both large and small molecules. The targeting mechanism of the present invention provides significantly improved efficacy for the delivery of radioisotopes for diagnostic and therapeutic procedures. In the same manner of application, the present invention is also suitable for monitoring therapeutic progress.

One advantage of the present invention is that a combination of imaging agents or therapeutic agents can be loaded into the virion derived nanoparticle. A further advantage is that the nanoparticle of the present invention is capable of targeting its radioactive tracer to specific cell receptors of tumor cells providing a precise delivery method for improved imaging differentiation. Additionally, because of the ability of the protein nanoparticle to deliver the radioactive isotope near the cell nucleus, the effect of radiation in the cell DNA is enhanced 1000 times and thus the efficacy is significantly improved.

The virion derived nanoparticles of the present invention may target all NCI-60human tumor cell lines to include: lung, colon, ovarian, renal, melanoma, CNS, hematologic, prostate, and breast.

Further aspects of the present invention relate to methods and compositions for producing virion derived, (e.g., papilloma virus (PV)-derived) protein nanoparticles containing one or more therapeutic or diagnostic agents. According to one aspect of the present invention, methods and compositions for encapsulating an agent within a virus like particle (VLP) may require an initial isolation and purification of capsid proteins produced in a host cell system (e.g. yeast, mammalian cell, insect cell, E. coli) and subsequent reassembly in vitro. Alternative the methods can include the initial purification of capsid proteins and/or VLPs produced in vitro without a host cell system.

Through the use of the present invention, the successful production of L1 and L2 full length capsomers in a bacteria host cell system (e.g. E Coli) with the correct folding and conformational structure has been accomplished. Still further, the present invention has made it possible to assemble these purified capsomers into virus like particles in vitro and to add radioisotopes without modifying the structure or stability of the final product.

The accompanying drawings, which are incorporated in and constitute part of the specification, illustrate various embodiments of the invention and together with the description, serve to explain the principles of the invention.

### BRIEF DESCRIPTION OF THE SEQUENCE LISTINGS AND DRAWINGS

FIG. 1 shows receptors over expressed by tumor cells that allow the specific binding and uptake of virus-like particles

FIG. 2 shows the transport of active molecules delivered by the virus-like particles into the cell nucleus

FIG. 3 shows a flow chart diagram of a preferred method for the preparation and purification of virus-like particles with a radioisotope

FIG. 4 shows a SDS-PAGE of fractions from purification of crude E. Coli homogenate by Heparin Affinity Chromatography

FIG. 5 shows results of HPV16 L2 detection by western blot of fractions collected from sucrose-gradient centrifugation.

FIG. 6 shows images of loaded VLP at a Direct Magnification of 50000× and a Printed Magnification of 272000@7 in.

(SEQ ID NO: 1) shows DNA sequence for E Coli L1X plasmid encoding papillomavirus mutant L1

(SEQ ID NO: 2) shows DNA sequence for E Coli L2 plasmid encoding papillomavirus L2

### DETAILED DESCRIPTION OF THE INVENTION

The present invention builds on the property that some viruses have of forming three-dimensional morphological subunits that make up the outer envelope of a viral shell, which may or may not correspond to individual proteins, called capsomers. Capsomers are pen tamers of the L1 capsid protein. Seventy-two assembled capsomers form the structure of the virus like particle of the present invention. The face of the virus like particle envelope consists of both L1 capsid proteins and L2 capsid proteins. It is described that the natural structure of the papillomavirus derived virus like particle has a stochiometry of L1:L2 of 10:1 (Trus et. al., Microsc Micronal 2005; 11(Suppl 2): 642-643). It is this aspect of the capsid structure and L1:L2 ratio that has been re-purposed to create nanoparticles that may be chemically modified, disassembled, loaded, and reassembled to deliver diagnostics and therapeutic payloads to cells and tissues of interest.

The terms “VLP”, “virion derived nanoparticles”, and “nanospheres” will be used interchangeably throughout the specification and claims, and refers to the capsid protein viral envelope as described above. VLP's are morphologically and antigenically similar to authentic virions. VLPs lack viral genetic material (e.g., viral nucleic acid), rendering the VLP non-infectious.

According to one aspect of the present invention, the ratio of capsid proteins to reaction volume may be manipulated to achieve greater loading capacities based on the chemical characteristics and the molecular size of the imaging agent or therapeutic agent or any combination thereof. This aspect of the invention incorporates the surprising discovery that varying the ratio of L1/L2 proteins, increasing the L1:L2 ratio to 5:1 (versus the wild type 10:1) is feasible and can create stable structures that can improve the delivery efficiency of radioisotopes to traffic to the nucleus region of a cancer cell and increase its efficacy by shortening the distance of the gamma radiation between the isotope and the target DNA.

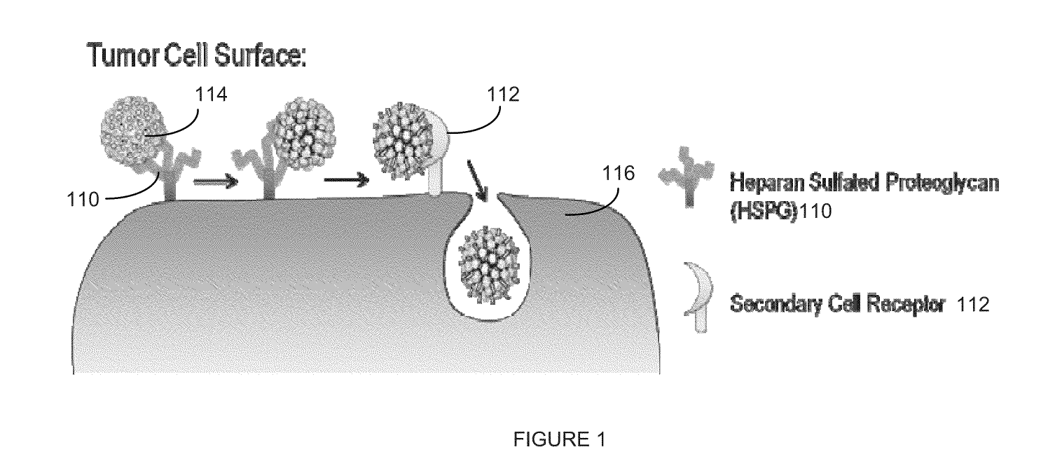

With reference now to FIG. 1, an illustration showing how the VLP nanospheres of the present invention might interact with tumor cell surface will now be discussed. As shown in FIG. 1, Heparan Sulphate Proteoglycans HSPG 110 and cell-surface receptors 112 are targeted by the present invention for reception of the VLP nanospheres with a radioactive pay load for diagnosis or therapy. The VLP nanospheres 114 may interact with specific HSPG 110 and cell-surface receptors 112. As a first step of interaction, capsid proteins interact with HSPG 110. As these cell-surface proteins are over-expressed by cancerous tumor cells 116 as compared with normal cells, the VLP nanosphere will be more likely to infect the tumor cell 116.

The virion derived, nanoparticles of the present invention are engineered to have a selective mechanism of action to identify cancer cells while sparing normal cells. As shown in FIG. 1, the mechanism of action is related to the ability of certain viral proteins to identity a pattern of Heparan Sulfate Proteoglycans (HSPG) that is unique to certain tumors and metastases and a secondary growth factor receptor that is over-expressed in the same cell

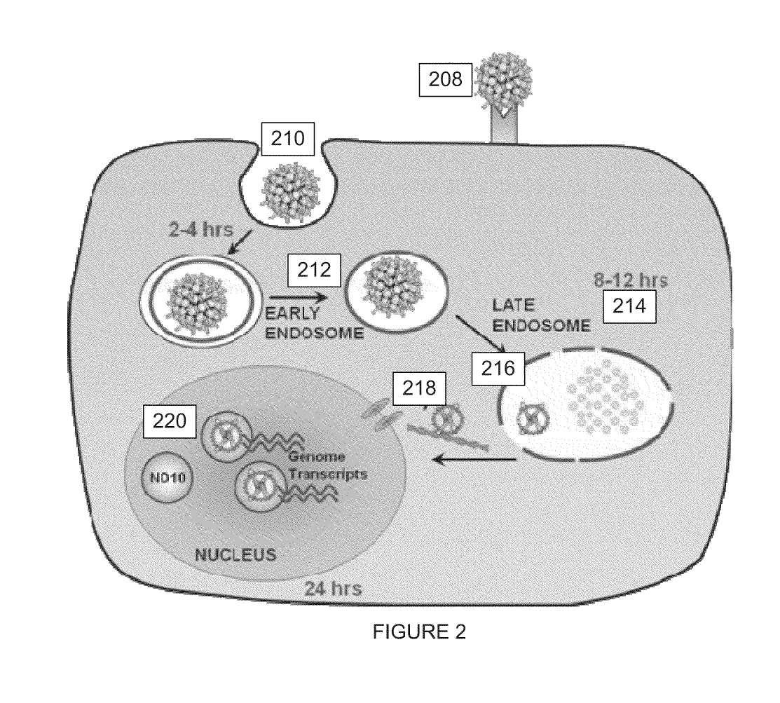

With reference now to FIG. 2, an illustration of radioactive isotopes transported in virion derived nanoparticles being internalized through a secondary receptor will now be discussed. As shown in FIG. 2, according to the present invention, after radioactive isotopes transported in virion derived nanoparticles attach to tumor cell receptors 208, they are endosomally internalized 210 through a series of stages which may be characterized as an early endosomal stage 212 from 2-4 hours after being internalized and a late endosomal stage 214 from 8-12 hours after being internalized. The medical imaging agent or therapeutic agent 218 is liberated within the cytoplasm as the nanoparticle disassembles 216 and is transported to the nucleus or perinuclear region 220.

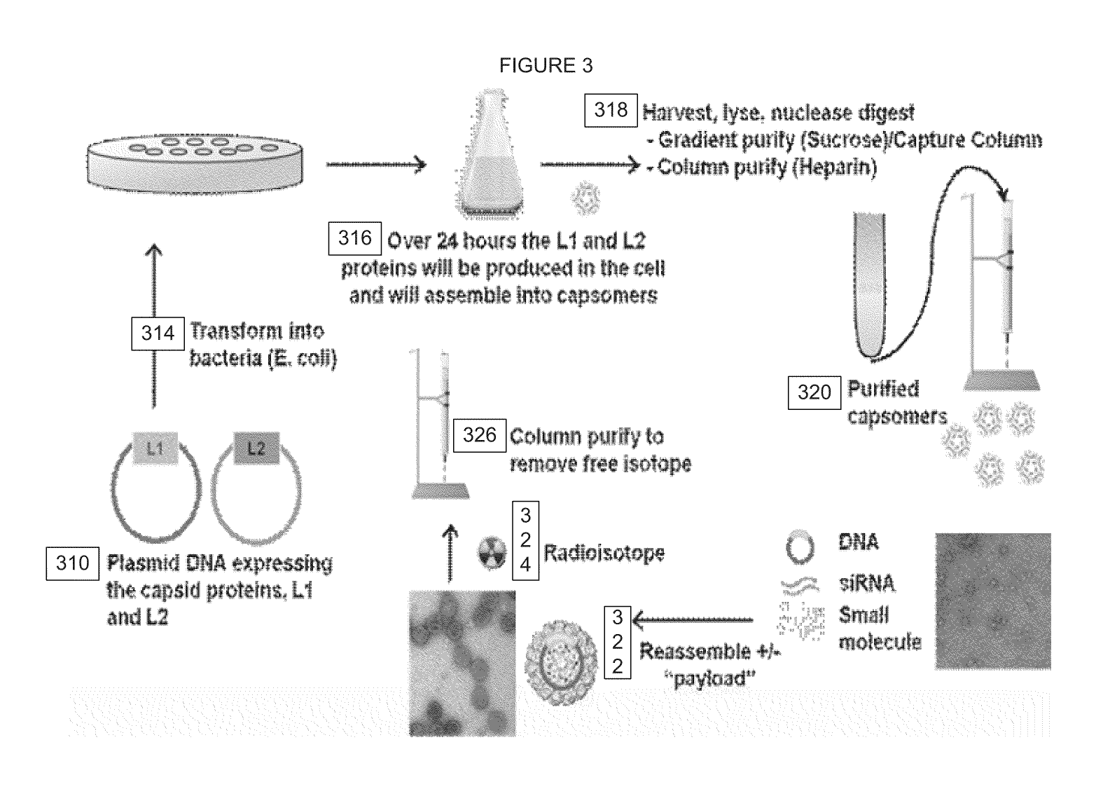

With reference now to FIG. 3, a preferred method, of loading nanospheres with a radioisotope will now be discussed. A recombinant DNA molecule containing a sequence encoding a papillomavirus L1 protein or a papillomavirus L2 protein or a combination of L1 and L2 proteins is codon optimized 310 and then transfected into a host cell (e.g. E coli) 312. Preferably, the plasmids may express papillomavirus L1 protein or L2 protein or a combination of L1 and L2 proteins in the host cell. Over the course of 24 hours the L1 and L2 proteins will be produced in the cell and will assemble into capsomers 316. The papillomavirus proteins expressed in the host cell are then preferably be harvested, lysed, nuclease digested and gradient purified (Sucrose)/Capture Column or column purified (Heparin) in the form of capsomers or smaller subunits 319. Purified L1 and L2 capsid proteins 320 may then be reassembled and combined with a short-lived radioactive isotope 322. The short-lived radioactive tracers may be linked through chemical reactions and/or compounds that help them attach to the protein structure (e.g. chelating agents) 324. Alternatively the radioisotopes may be attached to the interior of the nanoparticles through binding first to capsomers or structural sub-units. The capsomers including attached radioisotope payloads may then be column purified to remove any free isotopes 326 then administered to a subject by injecting into the bloodstream or into a localized compartment of the body.

Assembly of Particles

To combine the biological, pharmaceutical or diagnostic components to nanoparticles used as a carrier, the components can be associated with the nanoparticles through a linkage. By “used as a carrier associated with,” it is meant that the component is carried by the nanoparticles. The component can be dissolved and incorporated in the nanoparticles non-covalently through electrostatic interaction. Preferred and illustrative methods for creating, loading and assembling particles for use with the present are taught in following applications which are hereby incorporated by reference in their entirety: WO2010120266 entitled “HVP PARTICLES AND USES THEREOF;” WO2011039646, Nov. 24, 2010 entitled “TARGETING OF PAPILLOMA VIRUS GENE DELIVERY PARTICLES:”U.S. Provisional Application No. 61/417,031 entitled “METHOD FOR LOADING HPV PARTICLES;” and U.S. Provisional Application No. 61/491,774 entitled “PAPILLOMA-DERIVED PROTEIN NANOSPHERES FOR DELIVERING DIAGNOSTIC OR THERAPEUTIC AGENTS.”

According to one aspect of the present invention, methods and compositions have been developed for effectively encapsulating therapeutic and/or diagnostic agents within the structure of papilloma virus proteins (e.g., HPV proteins) that can be used for delivery to a subject. Virus like particles or Pseudoviruses for delivery of radioisotopes to tumors could be derived from viruses that have an inherent tumor tropism for example, Papillomavirus (PV) or herpes simplex viruses (HSV). Alternatively, other virus derived proteins which may be used as delivery agents within the scope of the present invention are not limited to but may include:, retroviruses, adenoviruses, adeno associated viruses, lentiviruses, poxivurses, bacteriophages, baculoviruses, and papillomaviruses. Some of these other viruses that are not tumor tropic can be modified by adding a target molecule to its structure.

According to a one aspect of the present invention, it may be useful to isolate L1 and L2 capsid proteins directly from host cells as opposed to disassembling VLPs that were isolated from host cells. L1 and L2 capsid proteins that are isolated directly from cells can be used during in vitro assembly reactions to encapsulate a therapeutic or diagnostic agent. This avoids the additional steps of isolating and disassembling VLPs. This also results in a cleaner preparation of L1 and L2 proteins, because there is a lower risk of contamination with host cell material (e.g., nucleic acid, antigens or other material) that can be contained in VLPs that are isolated from cells.

Isolated capsid proteins can then be used as described herein in a cell free system to assemble together with different payloads to create superstructures that contain a drug or diagnostic agent in its interior. Preferably, the payload capacity of the VLP can be precisely managed to deliver more exact radioisotope dosing to specifically targeted cell receptors. According to one aspect of the present invention, de-novo assembly of VLPs during the assembly procedure ensures formation of a larger percentage of loaded VLPs as opposed, to using already-formed VLPs for loading where a certain fraction can remain unloaded.

According to one aspect of the present invention, initial growth of the capsid proteins may be produced in a host cell system (e.g. yeast, mammalian cell, insect cell, Escherichia coli.) from independent expression nucleic acids (e.g., vectors, for example, plasmids) as opposed to both being expressed, from the same nucleic acid.

Preferably, the expression of L1 and L2 from independent plasmids allows the relative levels of L1/L2 VLP production to be optimized for different applications and to obtain molecular structures with optimal delivery properties for different payloads. In some embodiments, a variety of VLP structures can be produced to fit the needs of the different classes of payloads (e.g., radioisotopes, DNA, RNA, small molecule, large molecule) both in terms of charge and other functions (e.g. DNA binding domains, VLP inner volume, and endosomal release function). VLPs with a higher content of L2 protein will be better to bind nucleic acids (L2 contains a DNA binding domain) whereas VLPs with a smaller content of L2 protein will be better for other small molecules. VLPs with different ratios of L1:L2protein will have different inner volumes that will allow a higher concentration of drug to be encapsulated. According to one aspect of the present invention, the release of pay load into the cell may also be modulated. Alternatively, structures containing more L2 protein may have a higher ability to transfer nucleic acids intracellular. Preferably, different ratios of L1/L2 may be: 20:1, 10:1, 9:1, 8:1, 7:1, 6:1, 5:1, 4:1. However, other ratios may be used as aspects of the invention are not limited, in this respect.

The mode of administering the virion delivered short-lived, radioactive isotope may be by injection, inhalation or orally. Once introduced, accumulation in the targeted tissue may be viewed using a Positron Emission Tomography (PET Scan), Computerized Tomography (CT Scan), or any of a variety of other imaging techniques.

According to the present invention, some possible reactor radioisotopes that may be used may include: Bismuth-213, Chromunim-51, Cobalt-60, Dysprosium -165, erbium-169, Holium-166, Iodine-125, Iodine-131, Iridium-192, Iron-59, Lead-212, Palladium-103, Phosphorus-32, Potassium-42, Rhenium-186, Rhenium-188, Samarium-153, Technitium-99m, Lutetium-177, Sodium-24, Strontium-89, Xenon-133, Ytterbium-169, Ytterbium-177, Molydenum-99 as well as radioactive gold, caesium, or ruthenium. Some possible cyclotron radioisotopes that may be used according to the present invention may include: Carbon-11, Nitrogen-13, Oxygen-15, Fluorine-18, Cobalt-57, Copper-64, Copper-67, F-miso, 18-Fluro-2-deoxyglucose (FDG), Hg203, Gallium-67, Gallium-68, Germanium-68, Indium-111, Iodine-123, Iodine-124, Rubidium-82, Stronium-82, Thallium-201 chloride, Gadolinium-153, Yttrium-90 or other short-lived gamma emitters.

According to the present invention, delivery of alpha emitters for treatment preferably includes thorium-227, astatine-211, bismuth-212, lead-212, actinium-225, bismuth-213, fermium-255, radium-223 or terbium-149.

According to the present invention, delivery of auger emitters for treatment preferably includes ¹²⁵I ^{99m}Tc, ¹²³I, ¹¹¹In, ⁶⁷Ga, and ²⁰¹Tl, ¹¹⁹Sb

According to the present invention, delivery of gamma emitters for SPECT and PET-SCAN diagnostic and treatment preferably includes iodine-131, iodine-123, iodine-125, cobalt-60, iridium-192, lutetium-177, or palladium-103.

According to the present invention, delivery of beta emitters for treatment preferably includes iodine-131, Rhenium-186, Strontium-89, or Yttrium-90.

According to the present invention, positron emitters for PET preferably include fluorine 18 (used in FDG).

According to the present invention, delivery of contrast agents for MRI preferably includes gadolinium or selenium.

In addition to the medical imaging agent, the VLP of the present invention may be loaded with at least one therapeutic agent. Without limitation, the loaded therapeutic agent may include one or more of the following: a nucleic acid molecule capable of inducing RNA interference, inducers of DNA methylation, recombinant DNA coding for a toxic protein, modulators of gene expression, small molecules, proteins, antibodies or antisense molecules, micro RNA, biological therapies, immune-modulatory molecules, viral gene cassettes such as the myc-gene, viral proteins such as the P30 retrovirus protein or oncolytic virus proteins.

Most cancers are treated by external radiation which is not specific to tumor cells and damages normal cells. According to a further preferred embodiment of the present invention, the VLP loading method of the present invention may be employed against these cancers by allowing for the safe targeting and delivery of radiation treatments directly to the tumor cells nuclei enhancing the efficacy >1000 times and reducing the toxicity to surrounding healthy cells. Preferably, methods of radiation treatments, such as external beam radiotherapy, brachytherapy, and immunotherapy, or alpha radiotherapy, may be augmented or replaced by improved techniques of delivering radiation treatments with tumor targeted VLP that precisely deliver radiation treatments directly to the cells of interest without damaging normal cells.

Reduced Immunogenicity

An expression vector may be used to produce a mutant L1 or L2 protein with reduced or altered immunogenicity. In some embodiments, a mutant L1 protein (called L1*) is expressed along with L2 in a host system (e.g., a 293 cell system, E Coli). These can then be isolated and assembled as described herein to encapsulate a therapeutic or diagnostic payload. Preferably, according to one aspect of the present invention, loaded VLPs may be produced using L1 and/or L2 proteins that are modified to prevent cross reactivity with pre-existing antibodies against the viral proteins and/or to target the loaded VLP to particular organs or tissues (e.g., lung) or cells or sub-cellular locations that are non tumoral (e.g. dendritic cells).

Additionally, an expression vector may be used to produce a L1 or L2 protein from a distant related papillomavirus serotype (e.g. HPV 5) or from a non human papillomavirus (e.g. Bovine Papillomavirus, Mouse Papillomavirus, Macaque Papillomavirus, Rabbit Papillomavirus). In some embodiments a non human papillomavirus L1 protein or L1 and L2 protein is used to prevent cross reactivity with pre-existing antibodies against high risk human papillomavirus induced by vaccination.

Preferably, according to an aspect of the present invention, a VLP can be loaded with one or more medical, diagnostic and/or therapeutic agents, or a combination of two or more thereof. Accordingly, the methods described herein utilize PV-VLP that contain one or more variant capsid proteins (e.g., variant L1 and/or L2 capsid proteins) that have reduced modified immunogenicity or no cross reactivity with high risk HPV serotypes in a subject. Examples of variant capsid proteins are described in WO 2010/120266. The modification may be an amino acid sequence change that reduces or avoids neutralization by the immune system of the subject. In some embodiments, a modified PV-VLP contains a recombinant PV protein (e.g., a recombinant L1 and/or 1.2 protein) that includes one or more amino acid changes that alter the immunogenicity of the protein in a subject (e.g., in a human subject). A modified PV-VLP may have an altered immunogenicity but retains the ability to package and deliver molecules to a subject. The modification may be an electrolyte solution, pegylation or additional chemical modifications that reduce the particle recognition by the immune system. Such particles may be delivered to a subject without inducing an immune response that would be induced by a naturally-occurring HPV.

According to one aspect of the present invention, amino acids of the viral wild-type capsid proteins, such as L1 and/or L1+L2, assembling into the HPV-VLP, may be mutated and/or substituted, and/or deleted. These amino acids may be modified to enhance the positive charge of the VLP interior. Preferably, modifications may be introduced to allow a stronger electrostatic interaction of nucleic acid molecules or small molecules with one or more of the amino acids facing the interior of the VLP and/or to avoid leakage of nucleic acid molecules or small molecules out of the VLP. Examples of modifications are described in WO 2010/120266.

Production of L1 and L2 Capsomers

FIG. 3 provides an exemplary flow chart diagram of a preferred method for the manufacturing of papillomavirus capsomers according to an embodiment of the present invention. According to one aspect of the present invention two independent codon optimized L1 and L2 plasmids have been synthesized (SEQ 1, 2). Codon optimization has been designed to maximize bacterial (e.g. E Coli) expression. Plasmids have been transfected into E Coli cells and fermentation has been carried out as described in EXAMPLE 1 below. Further purification with sucrose gradient and size exclusion chromatography has been performed, to purify L1 and L2 capsomers as discussed in EXAMPLE 2 below. Expression analysis by Western Blot has confirmed, that the correct proteins have been obtained. Further in vitro modification of the solution including high salt concentration (e.g. 0.5M NaCl) and reduction of reducing agents (e.g. DTT) has demonstrated the ability of these capsomers to fold into virus like particles without losing their structural integrity or stability as discussed in EXAMPLE 2 below.

Loading with Radioisotopes

An aspect of the present invention is the successful loading of radioisotopes to the structure of the virus-like particles. Alpha emitters (Thorium-227, Actinium-225) can be attached to the structure of the proteins with chelating agents and Beta emitters (e.g. Iodine 131, Ytrium 90) can be attached by chemical conjugation to certain amino acids of the virus-like particles once it is assembled, or alternatively can be added to the amino acids of the capsomers and further reassembled to have a final product that comprises a VLP with a loaded radioisotope.

Aspects of the invention include the attachment of a bifunctional chelating agent in vitro (e.g. DOTA) either to the capsomers before reassembly or after reassembly to the outer structure of the protein and further addition of a radiometal (e.g Thorium-227) that will be chelated to form a stable radio labeled conjugate. Examples of DOTA chelating chemistries that can be used are very well described in the literature (Chappell et al, Synthesis and evaluation of novel bifunctional chelating agents based on 1,4,7,10-Tetraazacyclododecane-N,N_,N_,N_-Tetraacetic acid for radiolabeling proteins Nuclear Medicine and Biology 30 (2003) 581-595).

Aspects of the invention include the reassembly of virus-like particles from capsomers in vitro and further labeling with radioactive iodine, using a chemical reaction to link the iodine to the exposed tyrosines on the surface of the capsid proteins (EXAMPLE 3).

Alternatively the addition of radioisotopes to the VLPs can be achieved after VLPs have been further loaded with therapeutic agents utilizing a disassembly-reassembly method that has been described previously, for example in U.S. Pat. No. 6,416,945 and WO 2010/120266, incorporated herein by reference. Generally, these methods involve incubation of the VLP in a buffer comprising EGTA and DTT. Under these conditions, VLP completely disaggregate into structures resembling capsid proteins in monomeric or oligomeric form. A therapeutic or diagnostic agent, as described herein, may then be added and the preparation diluted in a buffer containing DMSO and CaCl₂ with or without ZnCl₂ in order to reassemble the VLP. The presence of ZnCl₂ increases the reassembly of capsid proteins into VLP. In some embodiments, one or more of these reassembly methods may be used to assemble capsid proteins to form VLPs that encapsulate one or more agents without requiring an initial VLP disassembly procedure, as described herein.

After isolation of L1 and/or L2 capsid proteins, VLPs may be loaded with one or more therapeutic agents and reassembled into loaded VLPs as described herein, the preparation diluted in a buffer containing DMSO and CaCl₂ with or without ZnCl₂ in order to reassemble the VLP. The presence of ZnCl₂ increases the reassembly of capsid proteins into VLP.

Certain ratios of a) Capsid protein to reaction volume, b) agent to capsid protein, and/or c) agent to reaction volume lead to agent-loaded VLP (VLP comprising entrapped agent) exhibit superior delivery of agent to target cells when compared to agent-loaded VLP prepared using previously described methods. VLP loaded with agents using the methods described herein, in certain embodiments, are able to deliver agent to 65%, 75%, 85%, 95%, 96%, 97%, 98%, or 99% of target cells. One non-limiting example of the improved method is exemplified in the Examples.

For example, a VLP may be loaded with a nucleic acid using a method comprising: a) contacting a preparation of capsid proteins with the nucleic acid in a reaction volume, wherein i) the ratio of capsid protein to reaction volume ranges from 0.1 μg capsid protein per 1 μl reaction volume to 1 μg capsid protein per 1 μl reaction volume; ii) the ratio of nucleic acid to capsid protein ranges from 0.1 μg nucleic acid per 1 μg capsid protein to 10 μg nucleic acid per 1 μg capsid protein; and/or iii) the ratio of nucleic acid to reaction volume ranges from 0.01 μg nucleic acid per 1 μl reaction volume to 10 μg nucleic acid per 1 μl reaction volume, and b) reassembling the capsid proteins to form a VLP, thereby encapsulating the nucleic acid within the VLP. In other embodiments, the ratio of HPV-capsid protein to reaction volume ranges from 0.2 μg HPV-capsid protein per 1 μl reaction volume to 0.6 μg HPV-capsid protein per 1 μl reaction volume. In yet other embodiments, the ratio of nucleic acid to HPV-capsid protein ranges from 0.5 μg nucleic acid per 1 μg HPV-capsid protein to 3.5 μg nucleic acid per 1 μg HPV-capsid protein. In yet other embodiments, the ratio of nucleic acid to reaction volume ranges from 0.2 μg nucleic acid per 1 μl reaction volume to 3 μg nucleic acid per 1 μl reaction volume.

The step of dissociating the VLP or capsid protein oligomers can be carried out in a solution comprising ethylene glycol tetraacetic acid (EGTA) and dithiothreitol (DTT), wherein the concentration of EGTA ranges from 0.3 mM to 30 mM and the concentration of DTT ranges from 2 mM to 200 mM. In certain embodiments, the concentration of EGTA ranges from 1 mM to 5 mM. In certain embodiments, the concentration of DTT ranges from 5 mM to 50 mM.

The step of reassembling of capsid proteins into a VLP can be carried out in a solution comprising dimethyl sulfoxide (DMSO), CaCl₂ and ZnCl₂ wherein the concentration of DMSO ranges from 0.03% to 3% volume/volume, the concentration of CaCh ranges from 0.2 mM to 20 mM, and the concentration of ZnCl₂ ranges from 0.5 μM to 50 μM. In certain embodiments, the concentration of DMSO ranges from 0.1% to 1% volume/volume. In certain embodiments, the concentration of ZnCl₂ ranges from 1 μM to 20 μM. In certain embodiments, the concentration of CaCl₂ ranges from 1 mM to 10 mM.

In certain embodiments, the loading method is further modified to stabilize the VLP, in that the loading reaction is dialyzed against hypertonic NaCl solution (e.g., using a NaCl concentration of about 500 mM) instead of phosphate-buffered saline (PBS), as was previously described. Surprisingly, this reduces the tendency of the loaded VLP to form larger agglomerates and precipitate. In certain embodiments, the concentration of NaCl ranges between 5 mM and 5 M. In certain embodiments, the concentration of NaCl ranges between 20 mM and 1 M.

Aspects of the invention are not limited in its application to the details of construction and the arrangement of components set forth in the preceding description or illustrated in the examples or in the drawings. Aspects of the invention are capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

### EXAMPLES

### Example 1

### Expression of L1 and L2 Capsid Proteins in a Bacterial Host Cell System

Aliquot 50 mL of Culture Medium, 50 μL of 50 mg/mL Kanamycin solution, and 50 μL of 100 mg/mL Ampicillin solution into a sterile disposable shake flask.

Place the shake flask and the glycerol stock vial E. coli BL21(DE3)-pET24-L1/pBAD-L2 in BCS, do not thaw the vial.

Inoculate the shake flask from the Intermediate Glycerol Stock vial: use a sterile 1-mL pipet to remove approximately 10 μL of frozen glycerol stock from the cryo-vial (avoid thawing) and immerse the tip of the pipette into the seed medium and stir briefly to inoculate.

Place the shake flask into the incubator shaker set at 30° C., 250 rpm and incubate overnight.

Measure the OD₆₀₀ of the overnight seed culture.

Aliquot 1 mL of 100 mg/mL Ampicillin solution and 1 mL of 50 mg/mL Kanamycin solution into each of the 1 L of culture medium in 2.8 L shake flasks.

Inoculate the shake flasks to an OD₆₀₀ of ˜0.1 with the appropriate amount of overnight seed culture.

Measure the OD₆₀₀ immediately after inoculation and periodically until the OD₆₀₀ approaches 4.

Expression Analysis

When the culture reaches an OD600 ˜4, remove 2×1 mL samples to microfuge tube, spin, remove supernatant, save as Pre-Induction sample. Transfer the culture into the 25° C. incubator to chill. Add 400 μL of 0.5M IPTG to the 1 L culture. Add 10 mL of arabinose solution to approximate 0.2% final concentration. After induction, place the shake flasks back into the incubator at 25° C. At and OD600 ˜8, take 3×1 mL samples to a microfuge tube, spin, remove supernatant, save as 1 hr post-induction sample pellet, and record OD600 in the appropriate table. Harvest each 1 L culture for clone 2 by centrifugation in ˜0.5 L aliquots; store the cell paste at −80° C. for use in downstream purification procedures.

### Example 2

### Purification of VLPs by Sucrose Gradient Centrifugation

Preparation of 10-65% linear sucrose gradient

Make a stock solution of 65% sucrose by dissolving 32.5 g of crystalline sucrose (Fisher cat. # 57-50-1) to a final volume of 50 ml sample buffer. Sample buffer used for VLP purification is 0.5M NaCl (American Bioanalytical cat. # AB01915) in sterile 1× PBS (Boston BioProducts cat. # BM 220S).

Make different concentrations of sucrose solution as described in Table 1 by mixing appropriate volumes of 65% sucrose stock solution (Step 1) in sample buffer.

TABLE 1

| | ml | |
|---|---|---|
| Final | 65% | ml |
| sucrose % | stock | buffer |
| 50 | 7.69 | 2.31 |
| 40 | 6.15 | 3.85 |
| 30 | 4.62 | 5.38 |
| 20 | 3.08 | 6.92 |
| 10 | 1.54 | 8.46 |

Gently overlay decreasing concentrations of sucrose (highest concentration at the bottom) in a Beckman Polyallomer centrifuge tube (Cat. # 326819). The volumes of different sucrose concentrations in the tube are as follows: 0.5 ml at 65%, 0.5 ml at 50%, 0.75 ml at 40%, 0.75 ml at 30%, 0.75 ml at 20% and 0.75 ml-1 ml at 10%.

Keep the gradient undisturbed at room temperature for 45 min. Gently load clarified lysate supernatant on top of the sucrose gradient without disturbing the layers below.

Centrifuge the tubes at 45,000 rpm at 4° C. for 2 hrs in a SW55Ti rotor (Beckman Coulter, Inc.).

Gently remove the tubes from the rotor and collect 0.2 ml fractions from bottom of the centrifuge tube. Analyze fractions by SD8-PAGE and BCA assay for total protein.

### Example 3

### Purification of VLPs Using Heparin HiTrap Column

After first centrifugation, if the homogenate is still turbid—re-centrifuged at 15,000 g for 30 min

Recover clarified homogenate from and store at −80° C. until use.

Add 0.01% Tween 80 to clarified homogenate.

Dialyze into PBS supplemented to 0.25 M NaCl, 2 mM DTT, 0.01% Tween 80, pH 7.4—overnight at 4° C. with three changes of buffer.

Equilibrate 1-mL HiTrap Heparin HP with 10 column volumes (CV) of dialysis buffer

Load entire volume of dialysed homogenate onto Heparin column at ˜0.1 mL/min

After loading, chase sample with ˜2 CV of dialysis buffer

Elute column with step gradient of increasing NaCl concentration—all steps contain PBS plus 1 mM DTT, 0.01% Tween 80-2.5 CV of each step: 0.4, 0.6, 0.8, 1.0 & 1.5 M NaCl

Collect 1.0 mL fractions of flow-through from loading and 0.5-mL fractions during elution

Determined absorbance of fractions at 260, 280 & 340 nm

Analyze load flow-through and NaCl gradient elution fractions by reducing SD8-PAGE on Bio-Rad TGX Any kD gels—stained with Coomassie R-250.

### Example 4

### Purification of VLPs by Size-Exclusion Chromatography

Preparation of an Agarose Gel Filtration Column

De-gas the DPBS-BSA solution by exposure to vacuum.

Clamp the column to a ring stand. Put the bottom cap on and add 5 ml of DPBS/0.5 M NaCl.

Remove the bottom cap to eject any bubbles. Recap and add more DPBS/0.5 M NaCl. Fill to near the top of the column.

Float a frit on the surface. Gently tap the frit to dislodge any air bubbles. Tap frit down to the bottom of the column using a 1- or 5-ml pipet (or the serum separator)

Remove the bottom cap and drain out most of the fluid.

Suspend the agarose beads by gently swirling and inverting the bottle. Pour bead slurry into the column. Fill the column to the rim.

Remove the bottom cap. Partially exchange the beads into room-temperature DPBS-BSA by repeatedly allowing the column to drip to near dryness then pouring on more DPBS-BSA.

Replace the bottom cap. Cover the top of the column with Parafilm. Suspend beads by repeated gentle inversion of the column. Return the column to the clamp and allow blocking and settling overnight at room temperature.

Remove Parafilm. Float a frit on the fluid surface and gently tap down to within a few mm of the bed surface.

Remove the cap from the bottom of the column. Wash the column with at least 10column volumes of DPBS/0.5 M NaCl.

Optional: If capsids are being purified out of crude cell lysate add 1 μl of Benzonase nuclease and incubate 10 to 30 min at 37° C. to digest any residual unencapsidated DNA.

Add 0.5 ml or less (i.e., less than 1/10 of the agarose bed volume) of clarified lysate (or capsids in Optiprep) to the washed agarose gel filtration column.

Apply 0.25 ml of DPBS/0.5 M NaCl to the top of the column. Collect column eluate in a siliconized 1.5-ml tube. Repeat this for a total of 12 0.25-ml fractions.

Screen fractions for encapsidated DNA and protein.

Regenerate columns for re-use by washing the column with 10 column volumes of DPBS/0.5 M NaCl, then exchanging into DPBS-BSA supplemented with 0.05% (w/v) NaN3or other preservative. Store the column at room temperature for several days.

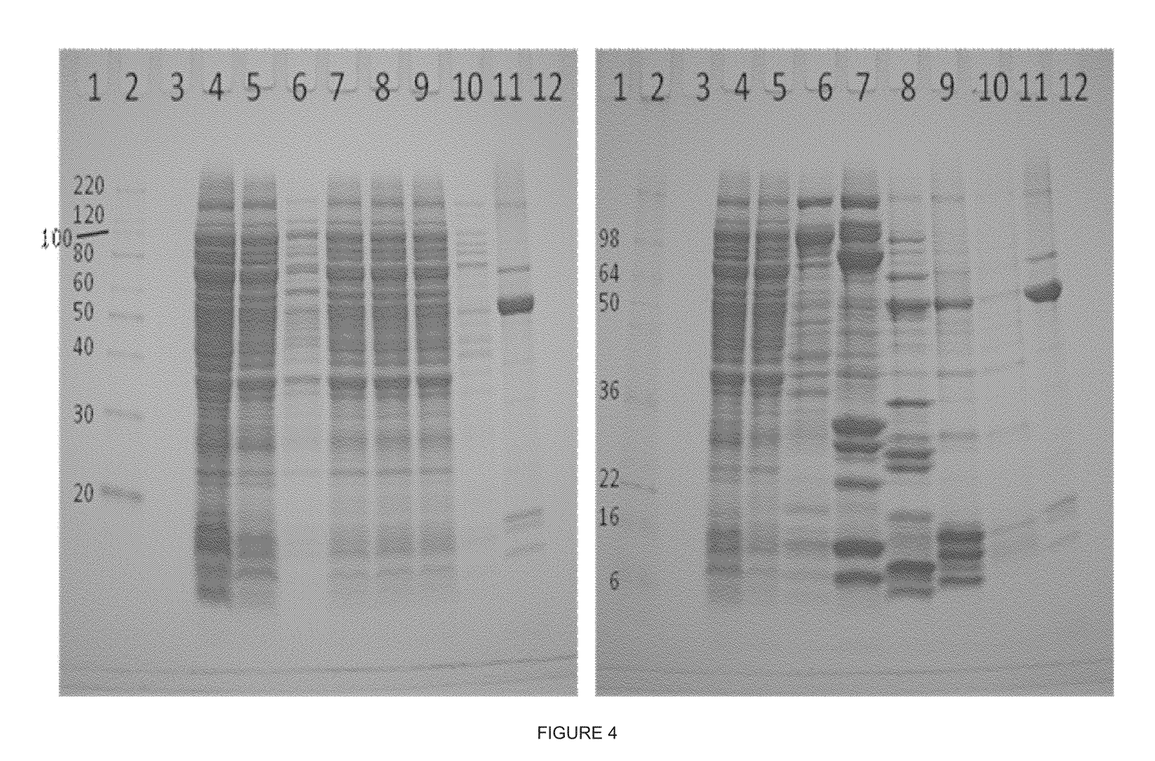

With reference now to FIG. 4, a SDS-PAGE, of fractions from purification of crude E. Coli homogenate by Heparin Affinity Chromatography, will now be discussed. As shown in FIG. 4, two gels: a left gel showing flow through fractions and a right gel showing Elution Fractions. The lanes on the left gel show the flow through fractions: lanes 1, 3, and 12 are sample buffer blank; lane 2 is a MW Std; lane 4 is a crude homogenate; lane 5 is a dialyzed homogenate (load); lanes 6 through 10 is load, flow thorough; and lane 11 is an L1/L2working standard. The lanes on the right gel show elution tractions: lanes 1, 3, and 12 are sample buffer blank; lane 2 is a MW Std; lane 4 is a dialyzed homogenate; lane 5 is load flow thorough F5; lane 6 is 0.4 M NaCl; lane 7 is 0.6 M NaCl; lane 8 is 0.8 M NaCl; lane 9 is 1.0M NaCl; lane 10 is 1.5 M NaCl; and lane 11 is L1/12 working standard.

Capsomers were purified from E. coli lysate and subjected to a single round of affinity purification on a heparin column. Electron microscopic images displaying spontaneously reassembled particles after reducing agent (DTT) concentration is lowered and salt (sodium chloride) concentration is increased.

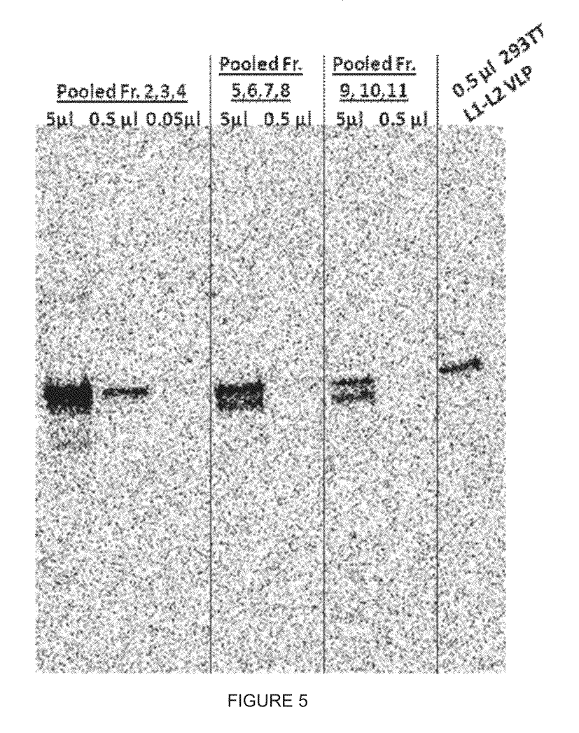

See FIG. 5 shows results of HPV16 L2 detection by western blot of fractions collected from sucrose-gradient centrifugation.

### Example 5

### Loading of VLPs with Iodine 131

Direct Iodination via iodo-beads: 2-3 Iodobeads (Pierce iodination reagent) rinse with 0.5 mls NaCl and discard ×3. Add Iodide (slightly basic) react 5-10 minutes.

Add 3-8 μg nanoparticle.

Monitor reaction with TLC.

Maximum observed yield −30% labeled particles after about an hour or two. Once labeling is complete, remove solution from beads, and purify.

TLC: Run plate all the way in ACN to move iodide, run plate halfway in H20 to move iodate.

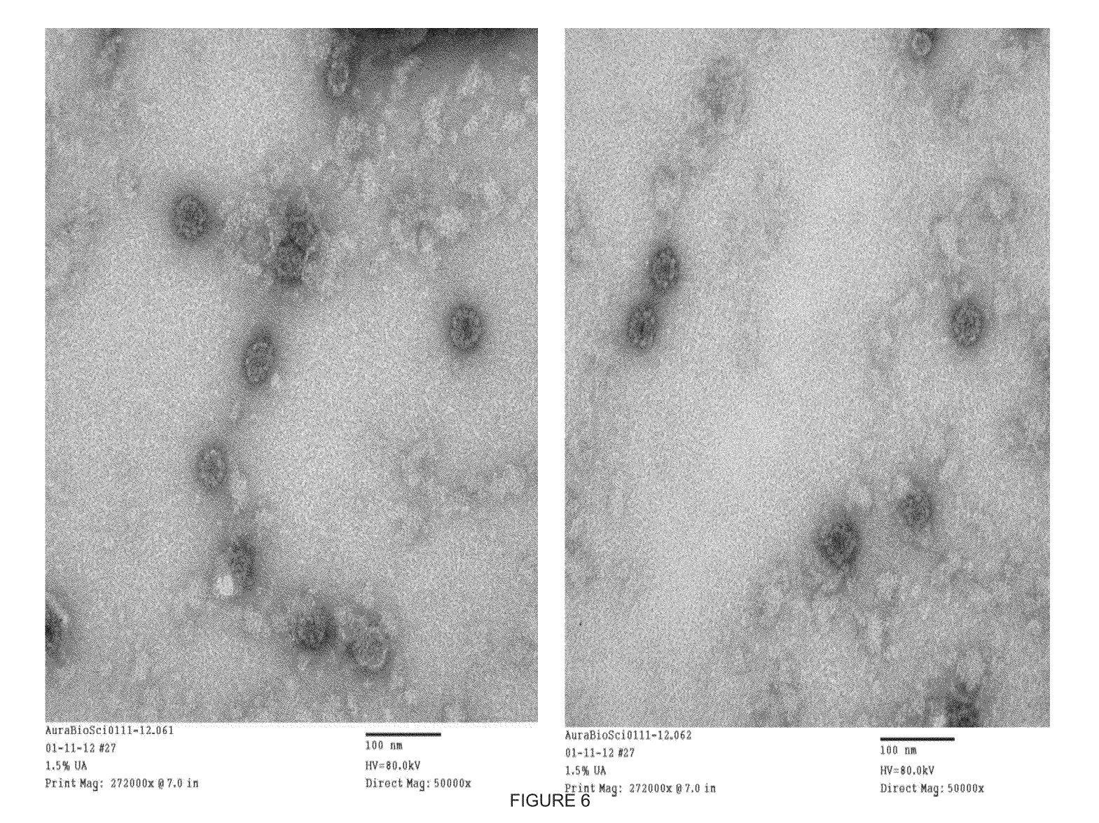

See FIG. 6, images of loaded VLP at a Direct Magnification of 50000× and a Printed Magnification of 272000@7 in.

While the above descriptions regarding the present invention contains much specificity, these should not be construed as limitations on the scope, but rather as examples. Many other variations are possible. Accordingly, the scope should be determined not by the embodiments illustrated, but by the appended claims and their legal equivalents. For example, the present invention applies to viral particles derived from any of a variety of virus proteins including those found within HPV, HSV, RSV, Polio, betaPV, Rotavirus and others. Further, the invention applies to all HPV types (1-120) and all types of HSV, RSV, Polio, betaPV, Rotavirus and other applicable virus types. Further, the invention applies to particles which are made with any of a variety of host cell systems including yeast, e-coli, insect, mammalian, or an in-vitro system such as e-coli. Further, the present invention applies to particles which are formed outside of a host cell system.

What is claimed is:

1. A method of making a virus-like particle, the method comprising:
constructing one or more recombinant DNA molecules containing a sequence encoding L1 or L2 capsid proteins or a combination of L1 and L2 capsid proteins;
transfecting one or more host cells with the recombinant DNA molecule(s);
expressing the L1 or L2 capsid proteins or a combination of L1 and L2 capsid proteins;
purifying the capsid proteins from the host cell(s);
combining the capsid proteins with a radioisotope, radiometal or radio-labeled molecule in vitro; and
assembling the capsid proteins to form radio-labeled virus like particles.

2. The method of claim 1, wherein the radioisotopes and/or the radioactive molecules are attached primarily to capsomers or smaller sub-units comprising L1 and L2 protein.

3. The method of claim 1, wherein the radioisotope and/or radioactive molecule are attached primarily to capsomers or smaller sub-units comprising L1 protein.

4. The method of claim 1, wherein the radioisotope and/or the radioactive molecules is added after reassembly of virus like particles.

5. The method of claim 1, wherein a bifunctional chelating agent is added to the structure of L1 and/or L2 proteins to enable the stable chelation of a radiometal.

6. The method of claim 1, wherein the bifunctional chelating agent is 1,4,7,10-tetraazacyclododecane-N,N_,N_,N_-tetraacetic acid (DOTA) or DOTA derivatives (IB4M-DOTA, C-DOTA, PA-DOTA, CHX-DOTA).

7. The method of claim 5, wherein the bifunctional chelating agent is added to the structure of L1 and/or L2 proteins after particle reassembly to enable the stable chelation of a radiometal, radioisotope or radiolabeled molecule.

8. The method of claim 1 wherein the radio-metal is an alpha emitter

9. The method of claim 1 wherein the radio-metal is Thorium 227, Actinium 225 or Astatin 211.

10. The method of claim 1 wherein the radio-metal is an Auger emitter.

11. The method of claim 10 wherein the Auger emitter is ¹²⁵I or ¹¹¹In

12. The method of claim 1, wherein at least one recombinant DNA molecule is codon optimized.

13. The method of claim 1, wherein at least one host cell is an E coli host cell.

14. The method of claim 1, wherein the recombinant DNA molecule(s) contain sequences encoding L1 and L2 that are regulated by different promoters

15. The method of claim 7, wherein the L1:L2 ratio is controlled to be less than 15:1.

16. The method of claim 7, wherein the L1:L2 ratio is controlled to be 5:1

17. A virus like particle, the particle comprising:
L1 and L2 capsid proteins, wherein the ratio of L1:L2 is less than 10:1; and
a radioisotope.

18. A virus like particle produced using the method of claim 1.


SEQUENCE LISTING (2 sequences)

SEQ ID NO: 1 - DNA, 6614 bp, Artificial Sequence
  DNA Sequence for E Coli L1X plasmid encoding papillomavirus mutant L1
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtcattacagtttacgcttcttacgcttcgcggtggtgctagtgctgctggtggtcggcgtggccttgcgcttgcccagggtaaacttcggttttgccttcagacccgcttgcagcaggaatttgcggcccagcggaaactggtccaaatcagccgagaatttctctttcagattgacctcccaaaaggtgtatttcttcagcggatcttctttcggtgccggtggggtatgcttctgacacgcgattgcctgggaggtaacaaaacggtacgtatcctccagcgtgccgccaggcggaggttgcagaccgaagttccaatcctccagaatcgtgctgttcatgctatgaatgtagctcatcacgtccgccgtcagggtgattttacacagctgaaaaatgaattgcagatcatattcttcgccgtgacgcagatattctttaaagttggtattcttataggtcggctcgctggtcgagatcgctgcacacaggctcatattcgtgctgcgcgtagtgtcaaccacggtaacaaacagttgattaccccagcaaataccattattgtgaccctgtgcacgttgcagccagtaaggcttattgaaaatctgagcatcgctagtaaccatgctgccgcttggcgtcggaaagtaattgctgttcgccaacgtcgcggtgctaccgctacctttgatgtacaggtcagtcggcacgttctcacccacggcacctgcgcgattgaacaggtgacggacgaacatttgctcgcgacgcaggtaaaagaacaggctgtcaccatacggttcgctgaccattttgatatagtccgggtacttgcagatagaggtgcaaatgtccaacggaacctcgctcttgttggcctgcaaggtggtaaagtccatcgcaccaaagcccgtatccaccatatcaccatcttgaatcaccgtattgatcagttccagcggtgggcaatcacccggattcaccgccacgttattgcacgggctacctttaccccagtgctcaccgatcggcggtttacaaccgatcaggcacagctgggtttgcttatagtccatcgaaatgcattcacgattatccacgcctgcgttcgcggcgtaggcagaagcgttctcggtatcgtccagtttgttcagcagcggatggccggagatgccgacgcccagcggctgaccacgaccaacctcaacgccgacacacgcccaaaccagacgctgcgtgtccgggttatagaagctggtgtccgggaaaccgaatttgttcgggtcacgcagatgaatgcggaacacacgatattgcaagccgctgaccttcggtaccagaattttgttgttgttcggtttcttaatcgggaaatacgggtgacccacggccaacaggcgggacgtacccgcgtggtagtagatattggtgcgcgcgacgtattcgtcggtagagacaaccttgctaactgggacaggcggtaagtacacggtcgcttcgctcgggagccacagggacattttttttatctcctttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 2 - DNA, 5386 bp, Artificial Sequence
  DNA sequence for E Coli L2 plasmid encoding papillomavirus L2
aagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgttttttgggctaacaggaggaattaaccatgagacacaaaagatcagccaaacgtacgaagagagcaagcgcgacccaactgtataagacctgcaaacaggcgggtacttgtccgcctgacatcatccctaaggttgagggtaagaccatcgcggatcaaattctgcaatacggcagcatgggcgttttctttggtggcctgggtattggtacgggtagcggcaccggcggtcgtaccggctacatcccgctgggcacccgcccaccgaccgccaccgatacgctggccccagtgcgtccgccgctgaccgtcgatccggttggcccgtccgacccgagcattgttagcctggtggaagaaaccagcttcattgatgcgggtgctcctacgagcgttccgtctatcccgccagacgtgagcggttttagcattacgacgagcaccgataccaccccggctattttggacattaacaacacggtgactaccgtgaccacccacaacaatcctacctttactgacccaagcgtgttgcaaccgccgaccccggcagaaacgggtggccacttcaccctgagcagctccaccatcagcacgcacaattatgaagagattccgatggacacctttatcgtatctacgaatccgaatacggtcacgagcagcaccccgattccgggctcccgtccggtcgcgcgtctgggtctgtactcccgtaccacccagcaggttaaagtcgttgacccggcgtttgttacgaccccgacgaagctgattacctatgacaatccggcctacgagggcattgacgttgataacaccctgtacttcagcagcaacgataatagcatcaatattgcaccggaccctgattttctggacatcgtcgcactgcaccgtccggcgctgacgagccgtcgcacgggtattcgttattcccgcatcggcaacaaacaaaccctgcgcacccgttcgggtaagtctatcggcgcaaaagtccattactattacgacctgtctaccatcgatccggcggaagagattgagttgcagacgattactccgagcacctacaccactacgtcccatgcagcgagcccgaccagcatcaacaatggtctgtacgacatctatgcggatgactttatcactgatacgagcaccacgccggtcccgagcgtgccgagcaccagcctgtcgggctatatcccggccaacaccacgattccgttcggtggtgcgtataacatcccgttggtgagcggtccagacatcccgatcaacattacggatcaggcaccgagcctgattccgatcgtcccgggtagcccacagtacaccatcattgctgatgcaggtgacttctacctgcatccgtcttactatatgttgcgtaaacgccgcaagcgtctgccgtacttcttctcggatgtgagcctggcggcgtaatgaattcgaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagag